(19) United States
(12) Patent Application Publication
(10) Pub. No.: US 2024/0033177 A1
(43) Pub. Date: Feb. 1, 2024

(54) APPARATUS AND COMPOSITION FOR INDUCING A CHRONIC STATE OF METABOLIC REDUCTION

(71) Applicants: ALMA MATER STUDIORUM - UNIVERSITA' DI BOLOGNA, BOLOGNA (IT); ISTITUTO NAZIONALE DI FISICA NUCLEARE, FRASCATI (IT)

(72) Inventors: Matteo CERRI, BOLOGNA (IT); Antonio ZOCCOLI, BOLOGNA (IT); Matteo NEGRINI, FORLI (IT); Marco DURANTE, DARMSTADT (DE)

(21) Appl. No.: 18/256,695

(22) PCT Filed: Dec. 10, 2021
(86) PCT No.: PCT/IB2021/061546

§ 371 Date: Jun. 9, 2023

(30) Foreign Application Priority Data
Dec. 11, 2020 (IT) 102020000030554

(51) Int. Cl. A61H 33/00

(52) U.S. Cl. CPC A61H 33/0095; A61H 2201/0285; A61H 2201/107; A61H 2230/505; A61H 2230/405; A61H 2230/045; A61H 2230/208

(57) ABSTRACT

An apparatus induces and autonomously maintains a state of hypometabolism for long time periods in people who would otherwise suffer from a significant deterioration of their vital functions and a shorter life expectancy, or people who will have to stay in a space environment for a long time. The apparatus, made up of a double enclosure, includes a chamber for housing one person, which is equipped with means for controlling the person's vital functions and the environmental conditions, with the possibility of autonomously modifying the environmental parameters and dosing the administration of substances according to data obtained from measured physiological variables. Outside the chamber there is an interspace containing a liquid for shielding any radiation. A non-invasive monitoring of the main vital functions includes oxygen consumption, carbon dioxide production, heart rate, hemoglobin saturation, temperature of various body districts, muscular tone, electroencephalographic activity, respiratory rate and exchanged respiratory volumes.

The present invention relates, in general, to devices for inducing a controlled state of metabolic reduction in humans, for medical and non-medical applications.

In the general medical field, it is known to subject patients to acute treatments aimed at obtaining a metabolic reduction (therapeutic hypothermia) in cases wherein high metabolic rate organs, like the heart or the brain, are in a state of difficulty in being supplied with energetic substrata (e.g. due to cardiac arrest, ictus etc.). Therapeutic hypothermia is induced by exposure to low ambient temperature and infusion of cold solutions. Since the human body, like the body of all mammals, is homeothermic, it is possible in these cases to observe the activation of compensatory physiological responses intended to counter the induction of therapeutic hypothermia.

Such compensatory responses may paradoxically lead to increased metabolism, in which case they will have to be pharmacologically suppressed. For this reason, therapeutic hypothermia is currently limited to just a few degrees of body cooling (not below 34° C.) and just a few days of treatment.

Therefore, therapeutic hypothermia cannot be used for long time periods, e.g. as may be required to extend the life of patients awaiting a transplant, nor can it be used for applications related to, for example, space exploration, quarantine, or protection against radiation damage, or anyway for any application other than acute medical-surgical ones.

Thus, solutions are needed, which may be alternative to those currently available, for significantly reducing metabolism for extended periods of time.

In light of this brief examination, it can be stated that the main technical problem at the basis of the present invention is to fulfil the current need for at least one solution to be used as an alternative to the techniques currently known in the art, in order to induce therapeutic hypothermia conditions in patients without the above-described contraindications.

In other words, the invention aims at providing an apparatus and/or a combination of substances adapted to prevent the onset of those contraindications which limit the techniques currently known in the art, such as sedation, anaesthesia, or the like, which are mostly used in surgery and intensive care and, anyway, for acute conditions.

The idea that solves this problem is to induce and maintain a chronic state of metabolic reduction in a person by means of a specially designed apparatus.

In this way, hypometabolism conditions are artificially reproduced which are comparable to those of lethargy in the mammals' world, through which metabolism can be drastically reduced even for long periods of time with no negative physiological effects.

Lethargy (or torpor, in technical terminology) is a behavioural state characterized by a metabolic reduction followed by a decrease in body temperature.

Here, hypothermia is a consequence of metabolic suppression and therefore, in contrast to therapeutic hypothermia, no body temperature compensation or defence mechanisms are triggered.

Multiple torpor episodes in succession lead to a state referred to as hibernation, which is typical of mammals such as squirrels, bears and hamsters.

In order to indicate induced conditions that simulate, even only partially, the essential elements of torpor/hibernation, several terms have been proposed in the scientific literature: pseudo torpor, suspended animation, torpor-like state, stasis, artificial hibernation, chemical hibernation/torpor.

The term “synthetic torpor” has been recently proposed, which will be used in this document. In particular, the use of synthetic torpor as a method of inducing and maintaining a long-term physiological stasis via the nervous system, associated with the use of vital support equipment, makes it possible to overcome the limitations of the current medical procedures.

Moreover, chronic synthetic torpor may be the first effective treatment for conditions that are at present incurable, like acute radiation syndrome.

The induction of a state of chronic synthetic torpor in humans permits:
a) extending the life of critical patients awaiting a transplant;
b) extending the time available for the explant of organs from donors in brain death conditions, thus optimizing the use thereof;
c) treating incurable diseases like acute radiation syndrome;
d) offering new treatment possibilities for all those conditions where metabolic reduction may be beneficial, such as status epilepticus or septic shock;
e) reducing the side effects of anticancer radiotherapy by increasing the radioprotection of healthy cells, thus also allowing the use of more aggressive radiotherapy protocols;
f) extending the ability of humans to explore the solar system beyond their current possibilities;
g) keeping in quarantine or isolation, if necessary, patients who have been exposed to various contaminants.

The features of the invention are specifically set out in the claims appended to this description. Such features as well as the effects and advantages of the invention will become more apparent in light of the following description of one possible embodiment thereof, provided herein with reference to the annexed drawings, wherein:

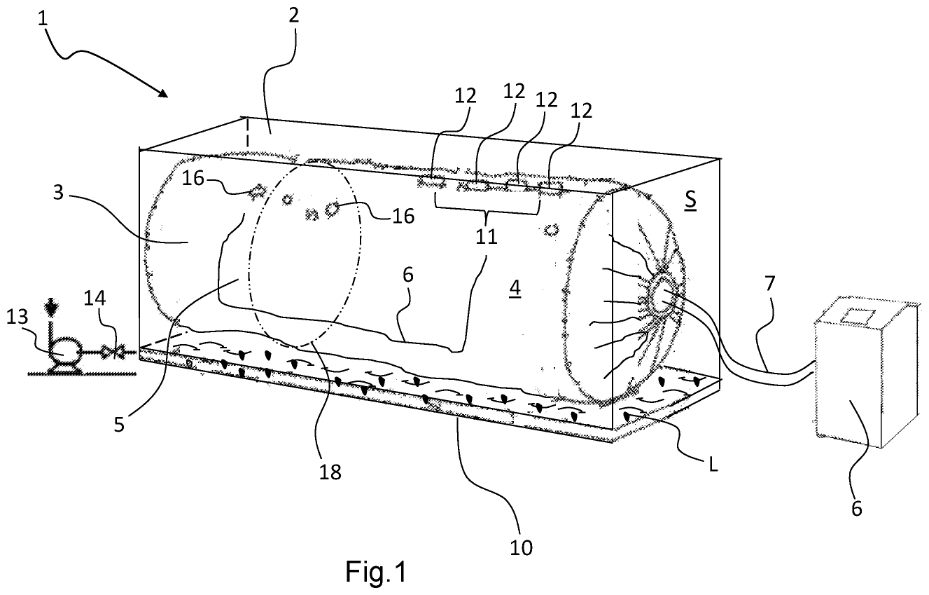

FIG. 1 shows a transparency view from the outside of an apparatus in accordance with the invention;

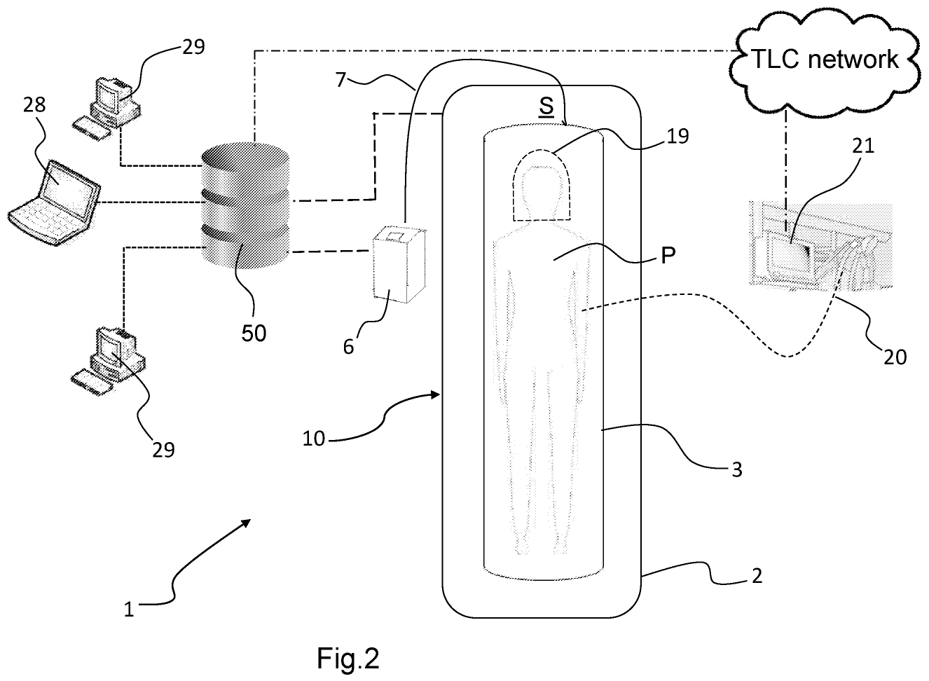

FIG. 2 shows a top view of the above apparatus, with a part thereof removed;

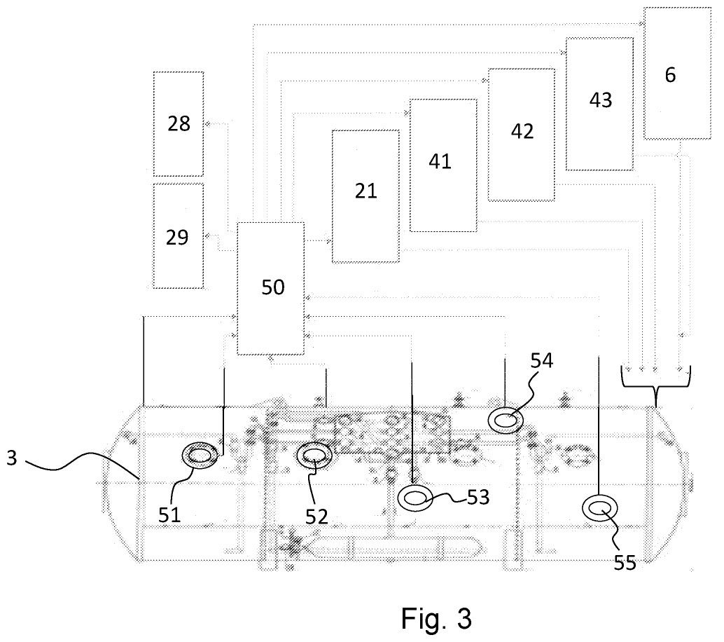

FIG. 3 is a diagram that illustrates the operation of the apparatuses of FIGS. 1 and 2.

With reference to the above-listed drawings, 1 designates as a whole an apparatus in accordance with the invention.

The apparatus comprises a capsule 10 for housing a person or patient P, configured with an external enclosure 2 with rigid or semi-rigid walls having a substantially parallelepiped shape, which contains another enclosure 3, made of flexible material, that houses the patient P.

The materials of the external enclosure 2 and internal enclosure 3 are the most appropriate ones for the purposes that will be further described hereinafter; in principle, however, any material can be used, including, for example, metal and/or plastic materials, moulded and/or laminated materials, films, textiles, etc. These details will not be further discussed herein, since they fall within the knowledge of those skilled in the art; instead, the focus will be on other aspects that are necessary or useful for understanding the invention.

In a preferred embodiment of the invention, the space S between the two enclosures 2 and 3 can be filled with a liquid L up to a predefined level; thus, according to the case, the space S may be filled either up to the top or to a fraction of the height of the capsule 10, e.g. ½, ¼, ⅔, ¾, etc.

This will depend on various circumstances such as, for example, the shape and dimensions of the enclosures 2 and 3, the fluid used for filling the space in between, the condition of the patient inside the capsule 10, etc.

The patient P is housed in a chamber 4 within the flexible enclosure 3, which is accessible through a sealed lateral opening 5; for this purpose, the opening 5 can be closed by means of an airtight slider-type fastener 6, or by means of a zip or Velcro® fastener or the like, sealed with impermeable thermoplastic material. Of course, any other solution suitable for this purpose may be used as well.

The atmosphere inside the housing chamber 4 is controlled in such a way as to ensure extended conditions of therapeutic torpor for the patient P.

To this end, the temperature inside the chamber 4 is thermostatically controlled through the use of heating and/or cooling means 11.

In accordance with a preferred embodiment, such heating/cooling means comprise a plurality of Peltier cells 12 distributed on the inner surface of the flexible enclosure 3.

As is known, Peltier cells are thermoelectric devices that generate heat in response to an electric voltage applied across them; the cells have opposing faces or surfaces ensuring thermal exchange with the surrounding environment; when the electric voltage is applied, such faces become hotter or colder, respectively.

In the context of the invention, by reversing the electric voltage of the cells 12 it is advantageously possible to reverse also the heating or cooling condition of their respective faces, so that they can either heat or cool the chamber 4 in the flexible enclosure 3 where the patient P lies.

The heating means 11 may nevertheless comprise other elements as well, which may be used as an alternative to or in combination with the Peltier cells 12.

Such elements may be electric resistors or infrared radiant panels, or the like.

It must be taken into account that the temperature and humidity of the air inside the chamber 4 can be controlled, whether wholly or partially, by means of appropriate air changes, which will have to constantly guarantee an adequate oxygen level for the synthetic torpor of the patient P. For this purpose, the internal enclosure 3 is connected, via a tubular duct 7, to an air conditioning and supplying unit 6.

Preferably, the unit 6 filters the air and sanitizes it (e.g. via application of UVA rays, ozone or the like), in addition to changing the air volume in the chamber 4 according to several detected parameters including, among others: air temperature, humidity and composition (i.e. O₂, CO₂, N₂, etc. percentage), conditions of the patient P, environmental conditions outside the apparatus 1.

The air change unit 6 as well as the other elements of the apparatus 1 (various sensors, the heating means 11, the Peltier cells 12, etc.) are managed by a control system of the apparatus 1, which will be further discussed below.

Therefore, the temperature of each section is constantly monitored by a (software) program handled by an electronic computer 50, such as a personal computer (PC), a server or the like, which can instantaneously modulate the degree of cooling or heating generated by the Peltier cells.

The chamber 4 that houses the patient P is also equipped with sensors for monitoring cardiac electrical activity, heart rate, hemoglobin saturation, respiratory rate, EEG, pO2 and pCO2, oxygen consumption, EMG in different muscular regions, ultrasounds directed to the renal vascular bed and to the heart.

Preferably, the capsule 10 is also equipped with defibrillation plates 16 and, at chest level, with an insulatable compartment 18 that may work as a negative-pressure respirator.

In one embodiment of the invention, the head of the patient P can be further enclosed in a helmet 19 made of semi-rigid material, into which a desired gas quantity can be supplied, whether by taking it directly from the environment or by administering a specific gas mixture. A venous route 20 may be attached to an automatic injector under control of the control unit 21. The control unit 21 is preferably controlled by an artificial intelligence managed by the computer 50, which can adjust the environmental parameters, the composition of the inhaled air, and the intravenous administration of fluids and drugs based on feedback obtained from the patient's physiological parameters.

In general, the capsule 10 is provided with the means necessary for housing a person P in metabolic reduction conditions for an extended time (a few weeks or longer).

The parameters of the various vital functions are preferably shown on displays 28, 29 outside the capsule 10.

In particular, as aforesaid, the atmosphere in the housing chamber 4 is kept in controlled sanitary conditions; therefore, air parameters such as temperature, pressure, composition (e.g. possible oxygenation), filtration and humidity are controlled in a systematic, continuous and automatic manner according to the feedback obtained from the patient's physiological variables.

In principle, it must anyway be pointed out that the structural and functional features of the apparatus 1 should preferably ensure at least the following functions:
i) controlling the ambient temperature in the chamber 4 and/or the body temperature of the patient P;
ii) non-invasive monitoring of the main vital functions: oxygen consumption, carbon dioxide production, skin temperature, central temperature, heart rate, hemoglobin saturation;
iii) possibility of immersing the body in liquids for protection against radiation-induced damages (water) or for protection of tissues exposed to perfluorodecalin or similar compounds and derivatives thereof;
iv) feedback control of the environmental parameters on the basis of the variations occurring in the patient's physiological parameters;
v) respiratory aid by negative-pressure respiration.

These functions are aimed at controlling and keeping the patient P in a state of artificial or synthetic torpor: in fact, in accordance with the teaching of the invention, by maintaining such a state it is possible to reduce a person's metabolism, much like hibernation in animals.

In this regard, it must be pointed out that the mechanism used by hibernating animals to enter the torpor/hibernation state has been unknown until not long ago.

Recently, a study conducted at the Applicant's laboratories (Hitrec et al., Sci. rep 2019) identified some brain areas which are essential components of the nervous network that induces metabolic suppression and cooling in animals.

Such regions, which are mostly, but not exclusively, concentrated in the hypothalamus, include areas such as the arched nucleus, the lateral hypothalamus, the dorsomedial hypothalamus, the preoptic area, the posterior hypothalamus, the paraventricular nucleus of the hypothalamus and the paraventricular nucleus of the thalamus, the periaqueductal grey, the parabrachial nucleus, the raphe pallidus, and afferent regions.

The study was conducted on laboratory animals, in particular mice. The mouse is a facultative heterotherm, i.e. an animal capable of entering a state of torpor as a means to improve its own survival expectancy in case of negative energetic balance, i.e. when it consumes more energy than is available.

This type of animal represents a model that is more similar to man than are seasonal hibernating animals, which are considered as subsequent adaptations of the ancestral hibernation phenotype.

As is known, some mammals can enter a state of hypothermic hypometabolism called torpor/hibernation because of the activation of a neuronal network composed of brain areas that control metabolism and body temperature.

The key area of this network is the Raphe Pallidus region, situated in the trunk of the encephalon.

It is proposed herein to modulate the activity of the Raphe Pallidus neurons in a direct or indirect manner in order to induce, in humans, a torpor-like state, hereafter referred to as synthetic torpor, that can last longer than one week.

Induction of synthetic torpor is effected by directly or indirectly modulating the Raphe Pallidus neurons by:
I) Inhibition of the Raphe Pallidus neurons by means of drugs exerting an inhibiting action on neuronal activity.

More in particular, it is proposed herein to use a combination of drugs comprising: GABA-A receptor agonists (e.g. muscimol); NMDA and non-NMDA glutamate receptor antagonists; M2 muscarinic receptor agonists, Y1 receptor agonists, ghrelin.

The concentration of the pharmacological combination may vary depending on the route of administration, which must be sufficient to reach an in situ concentration of 1 to 5 mM.

In accordance with the invention, several methods of pharmacological administration are possible, including:
a) Direct administration into the brain parenchyma by injection
b) Direct administration into the brain parenchyma by electrophoresis
c) Administration into the reference vascular district via intravascular catheter
d) Intraliquoral administration
e) Intraliquoral administration via peripherally inserted catheter (cisterna magna)
f) Epidural administration
g) Administration of liposomes containing the drug and use of resonance-guided converging ultrasound beams to break the liposomes in situ and release the drug
h) Systemic administration
i) Inhalation administration
j) Transdermal administration
II) Administration into the Raphe Pallidus neurons, by viral vector, of genetic material expressing DREADD inhibitory receptors, which can be chemogenetically activated by administration of clozapine N-oxide (CNO), olanzapine, salvinorin B (the administration routes being the same as those envisaged in I).
III) Administration of biological-action antibodies inhibiting the activity of the Raphe Pallidus neurons.
IV) Administration of drugs that suppress the metabolic pathways of the STAT3 gene. From the above description it is possible to understand how the invention works.

A patient P to be subjected to long-term metabolic suppression (e.g. because of lack of a compatible donor) is housed inside the capsule 10.

A state of torpor is induced in the patient by administration of one of the substances listed in the above options I), II) or III), dosed in accordance with several parameters, such as the patient's anamnesis and/or physiological conditions, or the time of permanence in the housing chamber 4 of the capsule 10.

The latter is then closed, and the required ambient conditions are maintained inside of it for the patient to remain in a desired state of torpor; for this purpose, the patient's vital functions are constantly measured and controlled by means of the electrocardiograph 41, the spirograph 42, the pulse oximeter 43 and all the other instruments necessary for performing the above-described functions.

As aforementioned, in accordance with a preferred embodiment of the invention, the operation of the apparatus 1 is controlled by an electronic computer 50, such as a personal computer or even a computer centre, to which the data relating to various operating parameters of the capsule 10 are transmitted, such as ambient temperature and pressure, composition and humidity of the air in the chamber 4 where the person lies.

Also the parameters pertaining to the patient's vital functions are monitored and transmitted to the computer 50: these include oxygenation, heart rate, body temperature, blood pressure.

As a function of the received data, the computer 50 will make any variations that may be necessary for the proper operation of the apparatus 1, based on a computation algorithm or program stored therein.

It should be noted that the computer 50, which is shown in the drawings to be close to the housing chamber 10, may also be in a remote position and connected to the rest of the apparatus 1 through a data transmission TLC network.

Such network is diagrammatically illustrated in FIG. 3, which shows, in the form of operator blocks, some elements that contribute to the operation of the apparatus 1.

Thus, for example, the chamber 4 may be equipped with temperature sensors 51, oxygen sensors 52, carbon dioxide sensors 53, a pressure switch 54, a hygrometer 55, which supply respective data to the control computer 50, which also receives data from the electrocardiograph 41, the spirograph 42, the pulse oximeter 43 and any other device used for monitoring the vital functions of the patient P.

The trend of the measured parameters can be displayed on the displays 28, 29. The latter are also connected to the computer 50, which processes and sends control commands to the units or devices used for maintaining the desired environmental conditions in the housing chamber 10.

Such units or devices may comprise the air change unit 6, the control unit 21, oxygen supply valves; filters; a pump 13 and valves 14 for supplying the liquid L into the empty space or interspace S of the capsule 10, and the like.

From the above description one can appreciate how the apparatus 1 and the substances or combinations of substances for inducing a state of torpor according to the invention make it possible to solve the above-discussed technical problem.

With this technology, in fact, it is possible to keep a person in a state of reduced metabolism for long time intervals, even for weeks, without prolonged use of substances that may be harmful for the body, such as those currently employed for the known sedation or intensive care treatments.

This eliminates, at the very root, all contraindications and side effects caused by such treatments, as previously described herein.

Lastly, it must be highlighted that the configuration of the capsule 10, which comprises an external enclosure 2 and an internal enclosure 3 between which an interspace S is defined which can be filled with a liquid L, makes it possible to shield radiations from the outside and to protect the person P lying in the housing chamber 4.

All the features of the invention described herein fall within the scope of the following claims.

1-12. (canceled)

13. An apparatus for inducing a chronic state of metabolic reduction comprising:
a capsule comprising an external enclosure and an internal enclosure, between which an empty space is defined.

14. The apparatus according to claim 13, wherein the empty space can be filled with a liquid.

15. The apparatus according to claim 13, wherein the external enclosure comprises rigid or semi-rigid walls, whereas the internal enclosure is substantially flexible.

16. The apparatus according to claim 13, wherein in the internal enclosure there is a housing chamber configured to receive at least one person, the apparatus comprising means for controlling vital functions of the at least one person and means for controlling environmental conditions in the housing chamber.

17. The apparatus according to claim 16, wherein the means for controlling vital functions of the at least one person comprises at least one of an electrocardiograph, a spirograph, a pulse oximeter, and a thermometer for detecting a body temperature.

18. The apparatus according to claim 17, wherein in the internal enclosure comprises Peltier cells for heating and/or cooling.

19. The apparatus according to claim 18, wherein the means for controlling vital functions of the at least one person and/or means for controlling environmental conditions within the housing chamber are adapted to perform one or more of the following functions:
i) controlling the ambient temperature in the housing chamber and/or the body temperature of the person;
ii) non-invasive monitoring of main vital functions: oxygen consumption, carbon dioxide production, skin temperature, central temperature, heart rate, hemoglobin saturation;
iii) possibility of immersing a body in liquids for protection against radiation-induced damages or for protection of tissues exposed to perfluorodecalin or to similar compounds and derivatives thereof;
iv) feedback control of environmental parameters on a basis of variations occurring in the person's physiological parameters;
v) respiratory aid by negative-pressure respiration.

20. The apparatus according to claim 19, comprising an electronic computer operatively connected to one or both of the means for controlling vital functions of the at least one person and the means for controlling environmental conditions within the housing chamber, for an execution of the functions.

21. The apparatus according to claim 20, wherein the housing chamber has a shape and dimensions such that the at least one person can be housed therein, and comprises at least one sealed access opening that allows maintaining a controlled atmosphere inside the housing chamber.

22. The apparatus according to claim 21, comprising a unit for supplying air into the housing chamber, with which elements are associated such as hoses, valves, dehumidifiers, filters, oxygen bottles and the like, for controlling the air conditions.

23. A combination of substances to be used for inducing a state of metabolic reduction, the combination of substances comprising:
GABA-A receptor agonists (e.g. muscimol);
NMDA and non-NMDA glutamate receptor antagonists; and
M2 muscarinic receptor agonists.

24. The combination according to claim 23, wherein a concentration varies depending on the route of administration, which must be sufficient to reach an in situ concentration of 1 to 5 mM.
